(19) United States
(12) Patent Application Publication
(10) Pub. No.: US 2018/0039885 A1
(43) Pub. Date: Feb. 8, 2018

(54) SATELLITE-BASED LOCATION IDENTIFICATION OF METHANE-EMITTING SITES

(71) Applicant: INTERNATIONAL BUSINESS MACHINES CORPORATION, Armonk, NY (US)

(72) Inventors: Conrad M. Albrecht, White Plains, NY (US); Josephine B. Chang, Bedford Hills, NY (US); Levente Klein, Tuckahoe, NY (US); Siyuan Lu, Yorktown Heights, NY (US); Fernando J. Marianno, New York, NY (US)

(21) Appl. No.: 15/228,753

(22) Filed: Aug. 4, 2016

(51) Int. Cl. G06N 3/08; G01N 33/22; G01N 33/00; G01N 21/69

(52) U.S. Cl. CPC G06N 3/08; G01N 21/69; G01N 33/225; G01N 33/0062

(57) ABSTRACT

Methods and systems for detecting emission sites include identifying a set of known emitters having visible features and a spectroscopic signature that correspond to sites that emit a substance to form a training set. A classifier is generated based on the training set. New emitters are identified based on the classifier, a spectroscopic signature map, and a map of visible features. An alert is provided responsive to the identification of a new emitter.

### BACKGROUND

### Technical Field

The present invention generally relates to pollution detection and, more particularly, to the identification of methane-emitting locations.

### Description of the Related Art

Methane emissions have a significant impact on climate change, as methane is a significant greenhouse gas and is the byproduct of a wide variety of industrial and agricultural processes. Methane emissions can be measured on-site, using static or mobile sensors that can probe the emission rate, but such solutions tend to be expensive and time consuming to implement on a large scale. This “bottom-up” approach monitors emissions at individual sites and sums their respective contributions, which produces an accurate result as long as all sites can be accounted for.

Alternatively, satellite data can be used to spectroscopically locate regions of high methane concentration. However, the resolution of this information is relatively coarse, for example having detection points that are 100 km by 100 km, aggregating emissions from all of the emitting sites within that area. Using this information to identify particular emitting sites is therefore very difficult.

### SUMMARY

A method for detecting emission sites includes identifying a set of known emitters having visible features and a spectroscopic signature that correspond to sites that emit a substance to form a training set. A classifier is generated based on the training set. New emitters are identified based on the classifier, a spectroscopic signature map, and a map of visible features, using a processor. An alert is provided responsive to the identification of a new emitter.

A method for detecting emission sites includes identifying a set of known methane emitters having visible features and a spectroscopic signature that is significantly above an average spectroscopic signature of methane at about 1.65 μm to form a training set. A classifier is generated based on the training set using machine learning. New emitters are identified based on the classifier, a spectroscopic signature map, and a map of visible features by applying the classifier in regions indicated by the spectroscopic signature map as having a higher than average spectroscopic signature of methane at about 1.65 μm, using a processor. An alert is provided responsive to the identification of a new emitter.

A system for detecting emission sites includes a training module configured to identify a set of known emitters having visible features and a spectroscopic signature that correspond to sites that emit a substance to form a training set. A machine learning module includes a processor is configured to generate a classifier based on the training set and to identify new emitters based on the classifier, a spectroscopic signature map, and a map of visible features. An alert module is configured to provide an alert responsive to the identification of a new emitter.

These and other features and advantages will become apparent from the following detailed description of illustrative embodiments thereof, which is to be read in connection with the accompanying drawings.

### BRIEF DESCRIPTION OF THE SEVERAL VIEWS OF THE DRAWINGS

The disclosure will provide details in the following description of preferred embodiments with reference to the following figures wherein:

FIG. 1 is a diagram of methane emitters and satellite data collection in accordance with the present principles;

FIG. 2 is a block/flow diagram of a method for identifying methane emitting sites in accordance with the present principles;

FIG. 3 is a block diagram of a system for identifying methane emitting sites in accordance with the present principles;

FIG. 4 is a block diagram of a processing system in accordance with the present principles;

FIG. 5 is a diagram of visible features of an emission site in accordance with the present principles; and

FIG. 6 is a diagram of visible features of an emission site in accordance with the present principles.

### DETAILED DESCRIPTION

Embodiments of the present invention use advanced image processing combined with machine learning to identify specific emission sites within satellite data. This combines two types of data—emission spectroscopy data that identifies regions with high emissions, and visual map data that is used to locate likely emitters within such regions. In this manner, the present embodiments provide the ability to locate specific emitters and to distinguish between types of emitter (e.g., between industrial and agricultural sources).

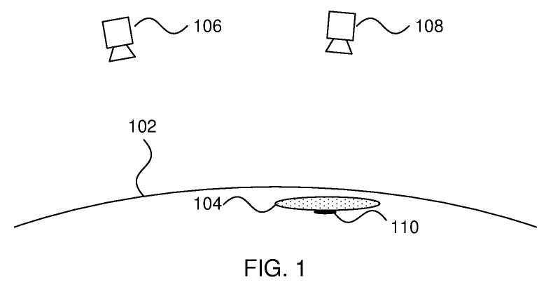

Referring now to FIG. 1, a view of a system of satellites is shown. A first satellite 104 and a second satellite 106 orbit the Earth 102. The first satellite 104 collects, e.g., visual data of the surface of the earth 102 to generate a map of the surface. The second satellite 106 collects data on other wavelengths, for example infrared radiation, from the surface of the earth 102. It should be understood that the first satellite 104 and the second satellite 106 are shown as being separate for the sake of explanation, but the functions of these satellites could also be incorporated in one single device with multiple imaging components.

In many cases the sites of interest are small and there is a need to identify them in satellite imagery. For example, gas well pads have typical dimensions of up to about 100 m by about 100 m, which would be just a few pixels in a typical Landsat satellite image that has a 30 m spatial resolution. Higher resolution imagery is needed to positively identify features in this range, such as that provided by the European Space Agency operated Sentinel satellite, which has a spatial resolution of 10 m, or private satellite providers like PlanetLab or Digital Globe that provide satellite imagery at a spatial resolution of 1 m or less. Thus two satellite data collection may be used to combine spectral information acquired at coarse spatial resolution and high resolution imagery to identify features on the well pads.

In particular, the second satellite 106 can collect emission spectroscopy data. In one specific example, the emission spectroscopy data can be collected using the Landsat 8 satellite, which has a number of different cameras sensitive to different wavelengths of light. Other satellites, such as the Greenhouse Gases Observing Satellite (GOSAT) or satellites that will be launched in the future may be used instead of Landsat 8 if they have the appropriate sensors installed.

In general spectroscopy refers to a type of measurement that collects information from some portion of the electromagnetic spectrum to identify substances. In this case, the absorption spectrum of a substance deals with the absorption and re-emission of light when it encounters the substance. Each substance will have certain discrete energy levels, generally representing the state of the electrons in a given atom or molecule. A substance will then absorb radiation that has energy that matches a difference between two energy levels, thereby causing a change in state. When the atom or molecule subsequently returns to its original state, the substance re-emits a photon having energy that matches the change in energy level. However, this re-emitted photon leaves in a random direction. As a result, the radiation that reaches a sensor through a medium is greatly attenuated at wavelengths that correspond to differences in energy levels in the medium. In this manner, particular substances in the medium can be identified by their characteristic absorption patterns.

Of particular interest in the present embodiments is the spectral band around 1.651 μm, which represents a strong absorption band for methane. It should be understood that, although the present embodiments focus specifically on methane, the present principles may be applied to any type of gaseous emission by collecting data from a spectral band appropriate to the gas in question.

As solar radiation at wavelengths of 1.651 μm reflects from the ground 102 toward the second satellite 106, it is absorbed by methane in the atmosphere 108. This causes less of the radiation to reach the satellite when there are significant concentrations of methane present. One camera in Landsat 8 in particular, designated shortwave infrared (SWIR) 1, covers the spectrum from about 1.57 μm to about 1.65 μm and has a spatial resolution of about 30 m. The data generated by this camera is therefore appropriate to use for the detection of methane, but it should be understood that any camera covering appropriate wavelengths, mounted on any satellite or any other device in space or low earth orbit, may be used instead. Since the bandwidth of the Landsat satellite is large, multiple gas species will influence the absorption of the solar radiation in the SWIR band. Beyond methane emissions, carbon dioxide, nitrous oxide, nitrogen dioxide, and water may impact the detected signal.

The data from the first satellite 104 and the second satellite 106 may be collected and compared at a location on the surface 102 to determine the specific locations of emitters 110 of methane concentrations 108. To accomplish this task, machine learning may be used based on a training set of manually identified, known emitter sites. Any well pad may have characteristics features. For example, well pads are typically square in shape, have no vegetation, and have infrastructure associated with the sites. Infrastructure may include, for example, compressors, pumps, storage tanks, and pools for liquid used in fracking. These features are easily recognizable from satellite imagery. Many of the well pad sites are in remote locations and there is a single road that goes toward the well pad. In many cases, these features can be recognized easily by a human operator by reviewing high-resolution satellite images.

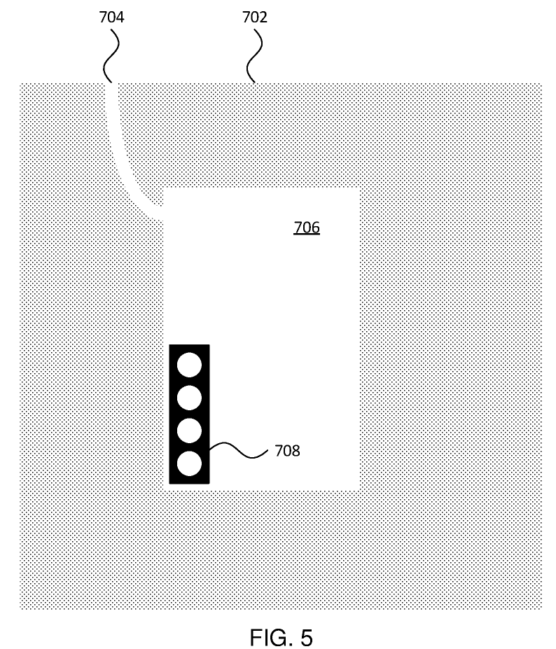

Referring now to FIG. 5, an exemplary well pad arrangement is shown, illustrating some of the visible features of a methane emitter that may be present in the satellite imagery. In this particular example, vegetation 702 is cleared away in a rectangular patch 706, with a single access road 704 leading to the site. In addition, there are structural features such as tanks 708 on the cleared patch 706 that are associated with methane emitters.

Since the number of sites that may be positively identified as having well pads is in the range of millions, these sites provide a well-defined data sets to train neural network and image processing techniques to identify characteristic features. The image processing may include, for example, geometric enhancements, edge detection, segmentation and feature extraction. Training data sets are used to identify such features.

Well pads are developed continuously, based on availability of the locations and the amount of gas that can be extracted from the ground. There is a delay between the moment when an application is submitted, permission to develop a gas well pad sites is granted, and the moment when the locations, characteristics and owner information becomes publicly available.

For those sites that are under development, but which have locations that cannot be determined from existing databases, real time satellite imagery may be used to pinpoint and validate locations that exist on the imagery, but not in public records. A selection of imagery of existing well pads may be used to identify such sights, with visible features being extracted and fed into the machine learning system.

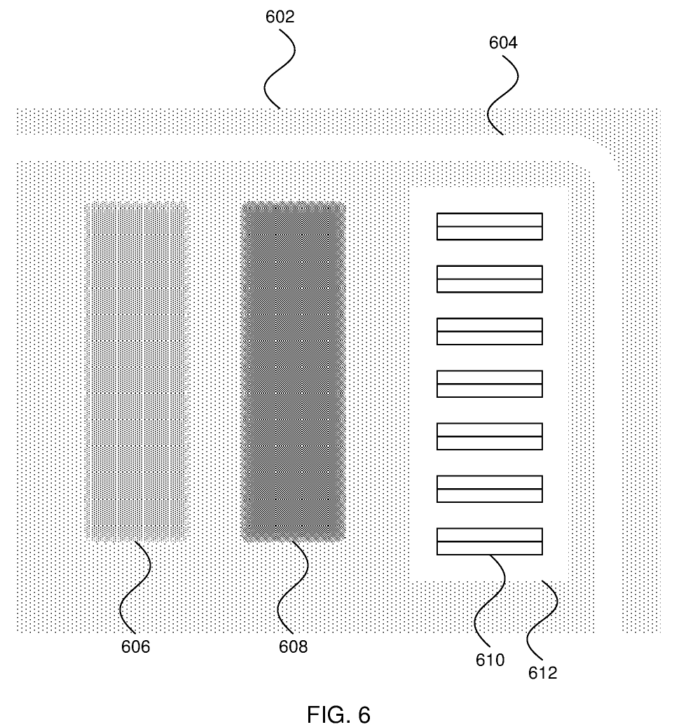

Referring now to FIG. 6, an exemplary farm arrangement is shown. The background 602 may be dirt or vegetation, with one or more access roads 604 running through the area. The visible area may include pools 608 or grazing areas 606 and may have one or more structures 610 constructed in a visibly demarcated area 612. Farms are often characterized by buildings 610 that are of uniform or similar shape and size and that are parallel.

The classifiers created by the present embodiments can distinguish between well pads, such as shown in FIG. 5, and farm area, such as shown in FIG. 6. The amount of methane emissions produced by a livestock farm is an indicator of the number of cows or other animals present on that farm. The methane level may also be an indicator of improper disposal of manure, which is an indicator in turn of whether environmental regulations are being followed.

Manual review can be used to eliminate images that may not be representatives or may be redundant in features. Furthermore, the data sets may be selected to be representative of the geographical characteristics of the sites, based on local regulations and requirements that may be implemented for these sites. One such regulation may be, for example, the minimum distance between a well pad and urban locations or human dwelling. If the criterion of minimum distance is not met, the data sets may be eliminated from the training data set.

The training data sets can be used to identify new sites that exist on recent satellite images that fully meet the criteria of the training data sets or meet them partially. Sites meeting partial criteria may be sites that are under development and that have, for example, the square shape and the connecting roads but show no compressor, storage tanks, etc.

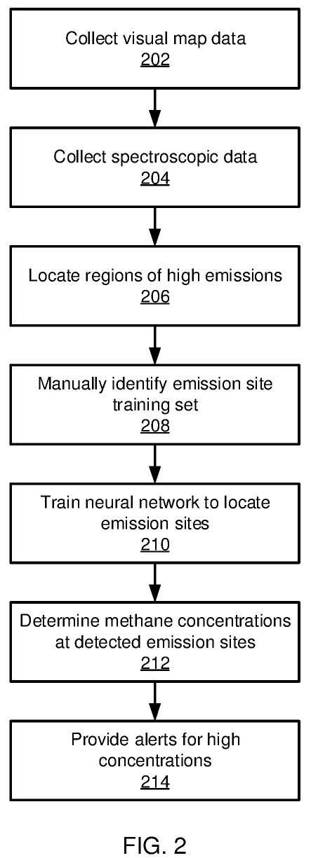

Referring now to FIG. 2, a method of locating emitter sites 110 is shown. Block 202 collects high-resolution map data. This data may be collected across a wide region or may be collected in a specific region of interest. It should be understood that commercial map data may be used for this purpose, as long as the map data provides images of sufficiently high resolution to identify structures and other relevant features. Block 204 collects spectroscopic data in the wavelengths of interest. As noted above, the present embodiments focus on methane and the spectrum around 1.65 μm, but it be understood that other wavelengths may be collected instead or in addition.

Using the spectroscopic data, block 206 locates regions of high emissions by determining areas where the signal (i.e., the amount of absorption) at 1.65 μm is significantly higher than average. In one exemplary embodiment, block 206 detects regions having a signal that is about 92% higher than average.

Within these regions, block 208 identifies specific emission sites and forms a training set. The location of each site may be extracted from the georeference sources. Additional features include size, shape, and orientation of the sites. Additional data layers may be leveraged including, for example, land owner information, prospecting information extracted from previous oil/gas surveys that indicate the availability of natural gas, soil properties, topography, vegetation, etc. Site identification can be performed manually by locating features in the high-resolution visual map in the region of high emission that correspond to likely emitters such as, e.g., an entry road, a generally rectilinear plot, storage tanks, a lack of vegetation, and the presence of industrial equipment.

A high spectral absorption, as detected by the satellite, may not be the only criterion used to identify a site. For example, livestock, swamps, landfill, and water treatment plants, may have spectral signatures that would indicate high methane emissions for that location. Other types of methane emitter, such as farms, may similarly be visually identified and demarcated based on features such as, e.g., rectilinear shape, size, fences for animal separation, ponds, parallel buildings, and proximity to roads. The operator marks these features on the map, for example by drawing a border around them. Different types of emitter can be distinguished by the operator, giving the system the ability to automatically determine not only the location of an emitter, but what kind of emitter (e.g., agricultural, industrial, or mining) is causing the emissions.

Block 210 then trains a machine learning system such as, e.g., an artificial neural network, using the training set. An artificial neural network is an information processing system that is inspired by biological nervous systems, such as the brain. The key element of artificial neural networks is the structure of the information processing system, which includes a large number of highly interconnected processing elements (called “neurons”) working in parallel to solve specific problems. Artificial neural networks are furthermore trained in-use, with learning that involves adjustments to weights that exist between the neurons. An artificial neural network is configured for a specific application, such as pattern recognition or data classification, through such a learning process.

The machine learning system thus uses supervised learning to build classifiers that enable the machine learning system to take unclassified data (e.g., new maps of regions having high concentrations of methane) and identify sites that are likely to be emitters. In block 202, the image is segmented and decomposed in individual components including, for example, infrastructure boundaries, the size of the elements composing the infrastructure, orientation, the number of elements within each image, the median distance between two well pads, and their spatial distribution. These identified sites may be ranked based on the strength of the spectroscopic signal above those sites and based on their geographical locations. Once a new site is identified, its characteristics will be compared with sites that are in close proximity of the identified sites. Block 210 thus automatically identifies emission sites based on correlations between the visual map and the spectroscopic data. In one example, the classifier may identify areas by providing a likelihood or confidence level that the areas are emitters, with those areas having a likelihood above a threshold value being identified as likely emitters.

Once sites have been identified as being likely emitters, block 212 determines the actual methane concentrations at these sites. Previous measurements of the methane emission and signal level can be correlated to quantify the signal detected in the satellite data. A scaling relationship can be used to assess the emissions at the moment of data acquisition from that particular site. Data from on-site monitoring of known emitters can be used to establish a correspondence between spectroscopic signal strength and actual methane concentrations, providing an inverse transformation that allows block 212 to determine methane concentrations based on spectroscopic data at the site and background spectroscopic data that represents the regional average. Block 212 also accounts for other gases, such as carbon dioxide, ammonia, nitrous oxide, carbon monoxide, etc., that may have some spectroscopic emissions within the band of emissions collected by the second satellite 106.

Once the emission concentrations have been determined by block 212, block 214 provides appropriate alerts regarding the concentrations. For example, if the concentration of a pollutant such as methane is above a threshold at a particular site, block 214 may notify an operator and initiate a response. The response may include, for example, informing the owner of the site of the emissions. Alternative responses may include sending a team to mitigate or eliminate the emissions. For emitters under the operator's control, block 214 may directly adjust operational parameters to reduce emissions below the threshold.

Block 214 may furthermore issue its alerts based on a comparison with government databases of emitters to ensure that registration compliance is in place, that environmental permits are in order, and that personnel have the proper training to work at the site. Owner information can be automatically extracted by accessing property and tax records, so that notices can be sent directly to the owners.

The methane concentration information and the associated emission sites are used to create a ranking of emission sites having the highest emissions. This may be used as the basis for a report of methane emissions for a region, with information about specific emission sites being provided for the purpose of emission reduction and regulatory enforcement. Additional information can be determined based on the methane emissions. For example, if a farm is located, the number of animals can be estimated based on the methane concentration. This information can furthermore be tracked over time, for example showing how the background methane concentration changes from season to season and from year to year.

Adding all the emission rate for all emission sites across a geographical area can provide an estimate for the methane level across that region. The methane will disperse, as it is lighter than air, but under constant emission rate the background level can be estimated. Knowing the methane level helps toward multiple goals: 1. Human safety for operators that need to visit a site, 2. Quantifying the total emission by site owners, 3. Enforcing compliance of maximum methane emissions by a company/site, and 4. Assessing dispersion of methane across an urban area as a public health hazard.

Adding up the emission rate may be misleading as the methane is dispersing. As such, an alternative method for determining the emission rate is to measure methane emissions across a larger area. Such measurements can be carried out by specialized satellites like GOSAT that estimates methane emission across an area of the order of, e.g., 100 miles by 100 miles. Within that 100 mile by 100 mile area there would be multiple emitters that contribute to the total emission level. The total methane level can be collectively assigned to all of the emitters and the additional information extracted from their distribution. The size of the emission can then be used to identify the collective impact of all methane emitters.

The methane level measured by GOSAT can be decomposed as the sum of multiple emitters and their emission rate. These emitters form the emission network of sources that are contributing to a certain regional methane levels. Using the satellite measurements and attributing the overall value to individual sites within that area is a top-down approach, where the number of emission site are unknown but their collective impact can be accurately measured. This constrain validates if the bottom-up approach and determines whether local measurements over- or under-estimates the emission rates. Once emission sites are identified and ranked based on emission rate, warning messages can be sent out to the company to fix the leaks, avoid sending people to the sites, and to take precautions due to health hazards within the area.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Reference in the specification to “one embodiment” or “an embodiment” of the present principles, as well as other variations thereof, means that a particular feature, structure, characteristic, and so forth described in connection with the embodiment is included in at least one embodiment of the present principles. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

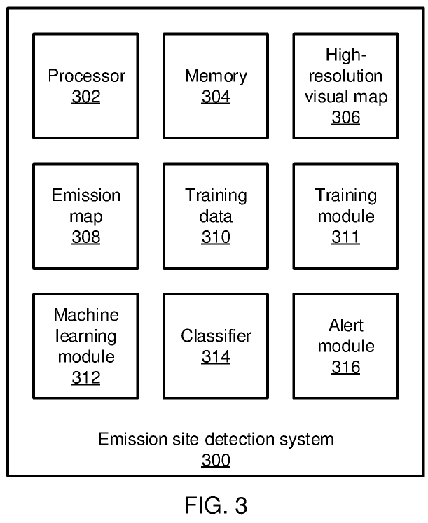

Referring now to FIG. 3, a system for emission site detection 300 is shown. The system 300 includes a hardware processor 302 and memory 304. The system 300 may also include one or more functional modules that may be stored in memory 304 and may be executed by the hardware processor. In an alternative embodiment, the functional module(s) may be implemented as one or more discrete hardware components in the form of, for example, application specific integrated chips or field programmable gate arrays.

The memory 304 stores high-resolution visual map information from the first satellite 104 and a spectroscopic emission map information 308 from the second satellite 106. Using the combination of these two data sets, an operator creates a training data set 310 by manually identifying locations in the visual map 306 that correspond to high emissions and that include one or more visible features that are associated with emission sites. The training set 310 may furthermore distinguish between different kinds of emission site (e.g., between agricultural and industrial). An operator works with the training module 311 to generate the training data set 310.

A machine learning module 312 uses the training data 314 to generate one or more classifiers 314. It is specifically contemplated that an artificial neural network may be used to generate the classifiers 314, but any appropriate machine learning process may be used instead. The machine learning module 312 then applies the classifiers 314 to new visual maps 306 and emission maps 308 to locate and identify sites having high emissions. An alert module 316 uses the information determined by the machine learning module 312 to provide alerts based on the emission concentrations, for example if the concentration exceeds a threshold.

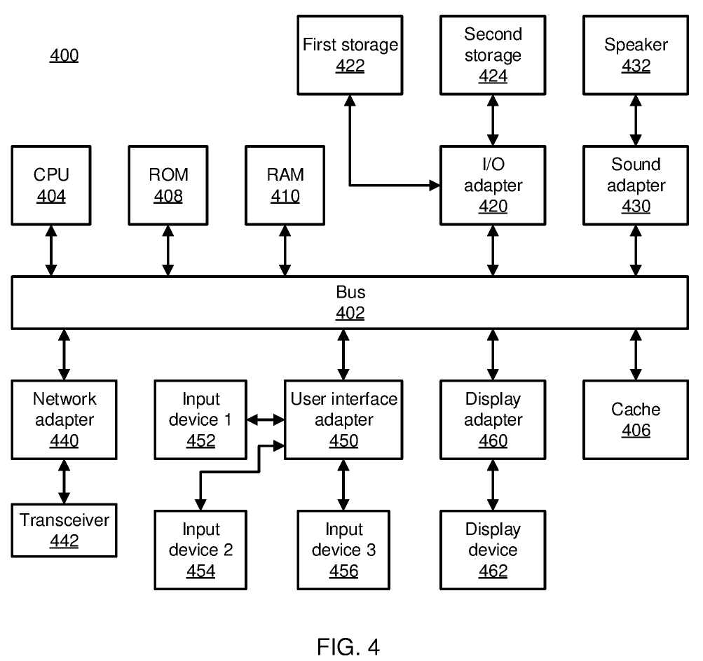

Referring now to FIG. 4, an exemplary processing system 400 is shown which may represent the emission site detection system. The processing system 400 includes at least one processor (CPU) 404 operatively coupled to other components via a system bus 402. A cache 406, a Read Only Memory (ROM) 408, a Random Access Memory (RAM) 410, an input/output (I/O) adapter 420, a sound adapter 430, a network adapter 440, a user interface adapter 450, and a display adapter 460, are operatively coupled to the system bus 402.

A first storage device 422 and a second storage device 424 are operatively coupled to system bus 402 by the I/O adapter 420. The storage devices 422 and 424 can be any of a disk storage device (e.g., a magnetic or optical disk storage device), a solid state magnetic device, and so forth. The storage devices 422 and 424 can be the same type of storage device or different types of storage devices.

A speaker 432 is operatively coupled to system bus 402 by the sound adapter 430. A transceiver 442 is operatively coupled to system bus 402 by network adapter 440. A display device 462 is operatively coupled to system bus 402 by display adapter 460.

A first user input device 452, a second user input device 454, and a third user input device 456 are operatively coupled to system bus 402 by user interface adapter 450. The user input devices 452, 454, and 456 can be any of a keyboard, a mouse, a keypad, an image capture device, a motion sensing device, a microphone, a device incorporating the functionality of at least two of the preceding devices, and so forth. Of course, other types of input devices can also be used, while maintaining the spirit of the present principles. The user input devices 452, 454, and 456 can be the same type of user input device or different types of user input devices. The user input devices 452, 454, and 456 are used to input and output information to and from system 400.

Of course, the processing system 400 may also include other elements (not shown), as readily contemplated by one of skill in the art, as well as omit certain elements. For example, various other input devices and/or output devices can be included in processing system 400, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized as readily appreciated by one of ordinary skill in the art. These and other variations of the processing system 400 are readily contemplated by one of ordinary skill in the art given the teachings of the present principles provided herein.

Having described preferred embodiments of satellite-based location identification of methane-emitting sites (which are intended to be illustrative and not limiting), it is noted that modifications and variations can be made by persons skilled in the art in light of the above teachings. It is therefore to be understood that changes may be made in the particular embodiments disclosed which are within the scope of the invention as outlined by the appended claims. Having thus described aspects of the invention, with the details and particularity required by the patent laws, what is claimed and desired protected by Letters Patent is set forth in the appended claims.

What is claimed is:

1. A method for detecting emission sites, comprising:
identifying a set of known emitters having visible features and a spectroscopic signature that correspond to sites that emit a substance to form a training set;
generating a classifier based on the training set;
identifying new emitters based on the classifier, a spectroscopic signature map, and a map of visible features, using a processor; and
providing an alert responsive to the identification of a new emitter.

2. The method of claim 1, wherein identifying the set of known emitters comprises identifying regions where the spectroscopic signature is significantly above an average spectroscopic signature.

3. The method of claim 1, wherein identifying new emitters comprises applying the classifier in regions indicated by the spectroscopic signature map as having a higher spectroscopic signature than average to locate sites that are likely emitters.

4. The method of claim 1, wherein the visible features include features associated with industrial or agricultural sites.

5. The method of claim 4, wherein the visible features comprise one or more of the group consisting of: an entry road, a generally rectilinear plot, storage tanks, a lack of vegetation, the presence of industrial equipment, bodies of water, fences, and a size or orientation of buildings.

6. The method of claim 1, wherein generating the classifier comprises learning the classifier by machine learning.

7. The method of claim 6, wherein machine learning comprises training an artificial neural network.

8. The method of claim 1, further comprising generating the map of visible features and the spectroscopic signature map by satellite.

9. The method of claim 1, wherein the substance is methane and the spectroscopic signature is an absorption signal at about 1.65 μm.

10. A non-transitory computer readable storage medium comprising a computer readable program for detecting emission sites, wherein the computer readable program when executed on a computer causes the computer to perform the steps of claim 1.

11. A method for detecting emission sites, comprising:
identifying a set of known methane emitters having visible features and a spectroscopic signature that is significantly above an average spectroscopic signature of methane at about 1.65 μm to form a training set;
generating a classifier based on the training set using machine learning;
identifying new emitters based on the classifier, a spectroscopic signature map, and a map of visible features by applying the classifier in regions indicated by the spectroscopic signature map as having a higher than average spectroscopic signature of methane at about 1.65 μm, using a processor; and
providing an alert responsive to the identification of a new emitter.

12. A system for detecting emission sites, comprising:
a training module configured to identify a set of known emitters having visible features and a spectroscopic signature that correspond to sites that emit a substance to form a training set;
a machine learning module comprising a processor configured to generate a classifier based on the training set and to identify new emitters based on the classifier, a spectroscopic signature map, and a map of visible features; and
an alert module configured to provide an alert responsive to the identification of a new emitter.

13. The system of claim 12, wherein the training module is further configured to identify regions where the spectroscopic signature is significantly above an average spectroscopic signature.

14. The system of claim 12, wherein the machine learning module is further configured to apply the classifier in regions indicated by the spectroscopic signature map as having a higher spectroscopic signature than average to locate sites that are likely emitters.

15. The system of claim 12, wherein the visible features include features associated with industrial or agricultural sites.

16. The system of claim 15, wherein the visible features comprise one or more of the group consisting of: an entry road, a generally rectilinear plot, storage tanks, a lack of vegetation, the presence of industrial equipment, bodies of water, fences, and a size or orientation of buildings.

17. The system of claim 12, wherein the machine learning module is further configured to learn the classifier by machine learning.

18. The system of claim 17, wherein machine learning comprises training an artificial neural network.

19. The system of claim 12, further comprising a memory configured to store the map of visible features and the spectroscopic signature map as supplied by one or more satellites.

20. The system of claim 12, wherein the substance is methane and the spectroscopic signature is an absorption signal at about 1.65 μm.
